(19) United States
(12) Patent Application Publication
(10) Pub. No.: US 2014/0323898 A1
(43) Pub. Date: Oct. 30, 2014

(54) System and Method for Monitoring Level of Dexmedatomidine-Induced Sedation

(71) Applicants: Patrick L. Purdon, Somerville, MA (US); Oluwaseun Johnson-Akeju, Dorchester, MA (US); Emery N. Brown, Brookline, MA (US); Michael J. Prerau, Somerville, MA (US)

(72) Inventors: Patrick L. Purdon, Somerville, MA (US); Oluwaseun Johnson-Akeju, Dorchester, MA (US); Emery N. Brown, Brookline, MA (US); Michael J. Prerau, Somerville, MA (US)

(21) Appl. No.: 14/261,188

(22) Filed: Apr. 24, 2014

Related U.S. Application Data
(60) Provisional application No. 61/815,614, filed on Apr. 24, 2013.

(51) Int. Cl. A61B 5/00

(52) U.S. Cl. CPC A61B 5/4821
USPC 600/544

(57) ABSTRACT

A system and method for monitoring a patient experiencing an administration of at least one anesthetic drug. In certain embodiments, the method includes arranging sensors configured to acquire physiological data from a patient and reviewing the physiological data from the sensors and an indication received from an input. The method also includes assembling the physiological data into sets of time-series data and determining, from the sets of time-series data, a first set of signals in a first frequency range and a second set of signals in a second frequency range, the first set of signals describing a transient oscillation signature and the second set of signals describing a target wave signature. The method further includes identifying, using the transient oscillation and target wave signatures, a degree of sedation consistent with the administration of the anesthetic drug, and generating a report indicative of the degree of sedation induced by the drug.

### CROSS-REFERENCE TO RELATED APPLICATIONS

This application is based on, claims priority to, and incorporates herein by reference in its entirety, U.S. Provisional Application Ser. No. 61/815,614, filed Apr. 24, 2013, and entitled “A SYSTEM AND METHOD FOR MONITORING LEVEL OF DEXMEDETOMIDINE-INDUCED SEDATION.”

### STATEMENT REGARDING FEDERALLY SPONSORED RESEARCH

This invention was made with government support under DP2-OD006454, DP1-OD003646 and TR01-GM104948 awarded by the National Institutes of Health. The government has certain rights in the invention.

### BACKGROUND OF THE INVENTION

The present disclosure generally relates to systems and method for monitoring and controlling a state of a patient and, more particularly, to systems and methods for monitoring and controlling a state of a patient receiving a dose of anesthetic compound(s) or, more colloquially, receiving a dose of “anesthesia.”

The practice of anesthesiology involves the direct pharmacological manipulation of the central nervous system to achieve the required combination of unconsciousness, amnesia, analgesia, and immobility with maintenance of physiological stability that define general anesthesia. More that 75 years ago it was demonstrated that central nervous system changes occurring as patients received increasing doses of either ether or pentobarbital are observable via electroencephalogram (“EEG”) recordings, which measure electrical impulses in the brain through electrodes placed on the scalp. As a consequence, it was postulated that the EEG could be used as a tool to track in real time the brain states of patients under sedation and general anesthesia, the same way that an electrocardiogram (“ECG”) could be used to track the state of the heart and the cardiovascular system. Despite similar observations about systematic relationships among anesthetic doses, EEG patterns and patients' levels of arousal made by other investigators over the next several decades, use of the unprocessed EEG in real time to track the state of the brain under general anesthesia and sedation never became a standard of practice in anesthesiology.

Hence, considering the above, there continues to be a clear need for systems and methods to accurately monitor and quantify patient states and based thereon, provide systems and methods for controlling patient states during administration of anesthetic compounds.

### SUMMARY OF THE INVENTION

Despite major advances in identifying common molecular and pharmacological principles that underlie effects of anesthetic drugs it is not yet clear how actions at different molecular targets affect large-scale neural dynamics to produce unconsciousness. Therefore, anesthesiologists are typically trained to recognize the effects of anesthesia and extrapolate an estimate of the “level” of anesthetic influence on a given patient based on the identified effects of the administered anesthesia. However, with increasing clinical use of anesthetics and the number of compounds with anesthetic properties growing, a scientific understanding of the operation of the body when under anesthesia is increasingly important. For example, a complete understanding of the effects of anesthesia on the brain over the continuum of levels of anesthesia is still lacking.

Tools used by clinicians when monitoring patients receiving a dose of anesthesia include EEG-based monitors, developed to help track the level of consciousness of patients receiving general anesthesia in the operating room and intensive care unit. Using proprietary algorithms that combine spectral and entropy measurements, these monitors typically provide feedback through partial or amalgamized representations of the acquired EEG signals. In addition, many monitoring systems attempt to quantify the physiological responses of a patient receiving a dose of anesthesia and, thereby, convey the patient's depth of anesthesia, through a single dimensionless index. Given that different drugs act through different neural mechanisms, and produce different EEG signatures, associated with different altered states of consciousness, existing approaches are qualitative at best. Consequently, existing EEG-based depth of anesthesia indices have been shown to poorly represent a patient's brain state, and moreover show substantial variability in underlying brain state and level of awareness at similar numerical values within and between patients. Not surprising, compared to non depth-of-anesthesia monitor based approaches, these monitors have been ineffective in reducing the incidence of intra-operative awareness.

In addition, standard depth of anesthesia monitors fail to properly characterize a depth of sedation. For example, at levels of dexmedetomidine sedation considered adequate using depth of anesthesia estimates provided by current monitoring systems, patients are readily aroused with sufficiently strong external stimuli. This is because EEG features associated with dexmedetomidine sedation are superficially similar to those encountered during general anesthesia.

The present disclosure overcomes drawbacks of previous technologies by providing systems and methods directed to monitoring and controlling a patient during administration of at least one anesthetic drug. Specifically, a novel approach is introduced for monitoring dexmedetomidine-induced sedation, using determined transient and low frequency oscillations present in acquired electroencephalogram (“EEG”) data to identify brain state signatures indicative of depth of sedation.

In one aspect of the present disclosure, a system for monitoring a patient experiencing an administration of at least one drug having anesthetic properties is provided. The system includes an input configured to receive physiological data from at least one sensor coupled to the patient and at least one processor configured to receive the physiological data from the input and assemble the physiological data into sets of time-series data. The at least one processor is also configured to determine, from the sets of time-series data, a first set of signals in a first frequency range and a second set of signals in a second frequency range, the first set of signals describing a transient oscillation signature and the second set of signals describing a target wave signature, and identify, using the transient oscillation and target wave signatures, a degree of sedation consistent with the administration of at least one drug having anesthetic properties. The at least one processor is further configured to generate a report indicative of the degree of sedation induced by the at least one drug having anesthetic properties.

In another aspect of the present disclosure, a method for monitoring a patient experiencing an administration of at least one drug having anesthetic properties is provided The method includes arranging at least one sensor configured to acquire physiological data from a patient, reviewing the physiological data from the at least one sensor and an indication received from an input, and assembling the physiological data into sets of time-series data. The method also includes determining, from the sets of time-series data, a first set of signals in a first frequency range and a second set of signals in a second frequency range, the first set of signals describing a transient oscillation signature and the second set of signals describing a target wave signature, and identifying, using the transient oscillation and target wave signatures, a degree of sedation consistent with the administration of at least one drug having anesthetic properties. The method further includes generating a report indicative of the degree of sedation induced by the at least one drug having anesthetic properties.

The foregoing and other advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will hereafter be described with reference to the accompanying drawings, wherein like reference numerals denote like elements.

FIG. 1 is a graphical illustration of example EEG data during administration of dexmedetomidine sedation.

FIG. 2A-B are schematic block diagrams of a physiological monitoring system.

FIG. 3A is an illustration of an example monitoring and control system in accordance with the present disclosure.

FIG. 3B is an illustration of an example portable monitoring system in accordance with the present disclosure.

FIG. 3C is an illustration of an example display for the monitoring and control system of FIG. 3A.

FIG. 4 is a flow chart setting forth the steps of a monitoring and control process in accordance with the present disclosure.

FIG. 5A is a flow chart setting forth steps of a method in accordance with the present disclosure.

FIG. 5B is a flow chart setting forth steps of a method in accordance with the present disclosure.

FIG. 5C is an example system for use in determining a brain state of a patient, in accordance with the present disclosure.

FIG. 6 is a graphical example indicating a relationship between probability of response, transient oscillation rate and transient oscillation power for EEG data acquired from a subject undergoing dexmedetomidine sedation.

FIG. 7 is a graphical example indicating a relationship between probability of response, transient oscillation rate and transient oscillation power for EEG data acquired from a subject undergoing dexmedetomidine sedation.

FIG. 8 is a graphical example indicating a relationship between probability of response, transient oscillation rate and transient oscillation power for EEG data acquired from a subject undergoing dexmedetomidine sedation.

FIG. 9 is a graphical example indicating a relationship between probability of response, transient oscillation rate and transient oscillation power for EEG data acquired from a subject undergoing dexmedetomidine sedation.

FIG. 10 is a flow chart setting forth the steps of an example of one clinical operation of the systems and method in accordance with the present disclosure.

FIG. 11 is a graphical example indicating a relationship between sedation and slow/delta (0.5 to 5 Hz) power.

### DETAILED DESCRIPTION

Dexmedetomidine has become an important drug in anesthesiology. It is utilized in the intensive care unit and in the operating room for sedation, and as an anesthetic adjunct. It allows patients to be placed in a state of sedation without respiratory depression, which is very desirable as this means that patients do not require airway instrumentation or ventilatory support. This helps to circumvent the increased morbidity associated with these procedures. Compared with propofol, one the most widely used anesthetic agent, patients are easily aroused when sedated with dexmedetomidine, and unlike propofol and benzodiezepines, dexmedetomidine is not typically used solely as a hypnotic agent. In addition, dexmedetomidine has analgesic properties, and induces a sedation state that resembles non-rapid eye movement (“NREM”) sleep.

Therefore, the present disclosure recognizes that NREM-like activity resulting from administration of drugs with anesthetic properties has important consequences with respect to systems and methods for monitoring and controlling sedation of a patient. As will be described, electroencephalogram (“EEG”) features similar to those exhibited during NREM sleep may be utilized to monitor sedation. In particular, “spindle”-like, or transient oscillation signatures, along with low frequency oscillation signatures, may be utilized to characterize the level of sedation.

Dexmedetomidine alters arousal primarily through its actions on pre-synaptic α₂-adrenergic receptors on neurons projecting form the locus ceruleus. Binding of dexmedetomidine to this G protein-coupled receptor hyperpolarizes locus ceruleus neurons and decreases norepinephrine release. The behavioral effects of dexmedetomidine are consistent with this mechanism of action. Hyperpolarization of locus ceruleus neurons results in loss of inhibitory inputs to the pre-optic area of the hypothalamus. The pre-optic area sends GABAergic and galanergic inhibitory projections to the major arousal centers in the midbrain, pons and hypothalamus. Hence, loss of the inhibitory inputs from the locus ceruleus results in sedation due to activation of these inhibitory pathways from the pre-optic area to the arousal centers. Activation of inhibitory inputs from the pre-optic area may be an important component of how NREM sleep is initiated. Sedation by dexmedetomidine is further enhanced due to the blockage of pre-synaptic release of norepinephrine leading to toss of excitatory inputs from the locus ceruleus to the basal forebrain, intralaminar nucleus of the thalamus and the cortex. The relationship between the actions of dexemedetomidine in the pre-optic area and the initiation of NREM sleep can explain the similarities in the EEG patterns between this anesthetic and those observed in NREM sleep.

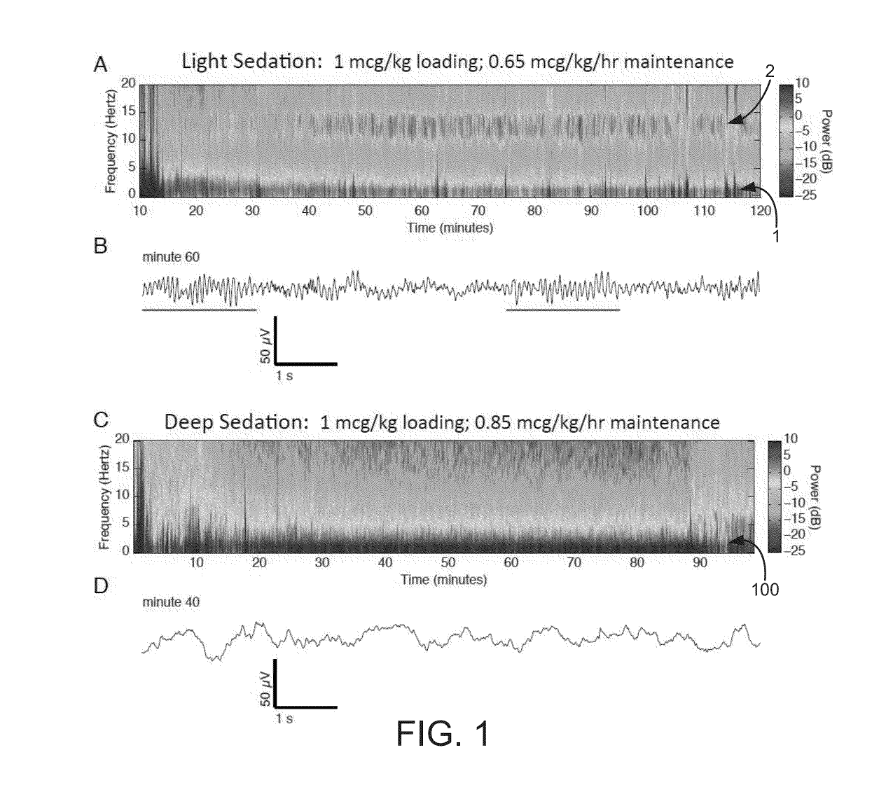

Referring specifically to FIG. 1, example EEG data for a patient undergoing dexmedetomidine sedation is shown using a spectrogram representation, illustrating power as a function of time for EEG signals in a range of frequencies. Specifically, when dexmedetomidine is administered as a low-dose infusion, it induces light sedation, meaning that with a minimal auditory, tactile or verbal stimulation, a patient can respond verbally. As shown FIG. 1A, observed features include a combination of low frequency oscillations 1, such as slow wave oscillations or delta wave oscillations, (with frequencies less than 6 Hz) and “spindles” 1, or spindle-like events, which are transient oscillations, generally in a frequency range of 9 to 16 Hz that occur in bursts lasting 1-2 seconds (FIG. 1B). In the spectrogram of FIG. 1A, the dexmedetomidine spindles 2 appear as streaks in the high alpha (9-12 Hz) and low beta (13-25 Hz) bands, occurring in a similar frequency range as alpha oscillations generated during propofol-induced anesthesia, but with much less power than alpha oscillations. It is noteworthy, that dexmedetomidine spindles 2 are reminiscent of signatures defining stage II NREM sleep. In addition, low frequency oscillations 1 are also apparent in the spectrogram of FIG. 1A, showing power close to zero frequency. On the other hand, when the rate of dexmedetomidine infusion is increased, spindles disappear and the amplitude of low frequency oscillations 1 increase (FIG. 1D), appearing as intense power in the low frequency band, such as slow wave or delta wave band, (FIG. 1C), which is considerably stronger than low frequency, such as slow wave or delta wave, oscillations power observed during administration of lower dose of dexmedetomidine. This EEG signature pattern of low frequency, such as slow wave or delta wave, oscillations 1 resembles features of NREM sleep stage III or slow-wave sleep.

As detailed below, the present disclosure takes advantage of signatures in physiological data, such as EEG data, acquired via sensors coupled to the patient during administration of at least one drug having anesthetic properties, providing a novel approach to monitoring and/or controlling sedation. That is, such patterns or signatures can be used as markers or indicators to determine a current and/or future state of the patient. Particularly with reference to dexmedetomidine sedation, systems and methods are described that can recognize spindle, or transient oscillation, signatures as well as low frequency oscillation signatures and use such to characterize a degree, or depth, of sedation.

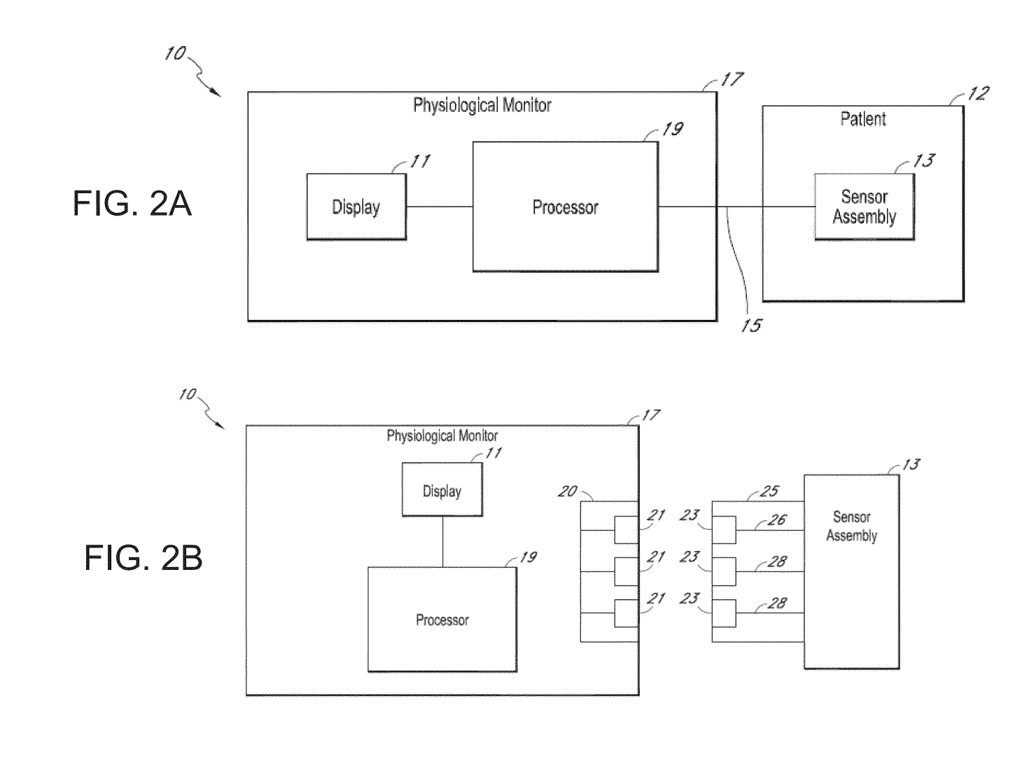

Referring specifically to the drawings, FIGS. 2A and 2B illustrate example patient monitoring systems and sensors that can be used to provide physiological monitoring of a patient, such as consciousness state monitoring, with loss of consciousness or emergence detection.

For example, FIG. 2A shows an embodiment of a physiological monitoring system 10. In the physiological monitoring system 10, a medical patient 12 is monitored using one or more sensors 13, each of which transmits a signal over a cable 15 or other communication link or medium to a physiological monitor 17. The physiological monitor 17 includes a processor 19 and, optionally, a display 11. The one or more sensors 13 include sensing elements such as, for example, electrical EEG sensors, or the like. The sensors 13 can generate respective signals by measuring a physiological parameter of the patient 12. The signals are then processed by one or more processors 19. The one or more processors 19 then communicate the processed signal to the display 11 if a display 11 is provided. In an embodiment, the display 11 is incorporated in the physiological monitor 17. In another embodiment, the display 11 is separate from the physiological monitor 17. The monitoring system 10 is a portable monitoring system in one configuration. In another instance, the monitoring system 10 is a pod, without a display, and is adapted to provide physiological parameter data to a display.

For clarity, a single block is used to illustrate the one or more sensors 13 shown in FIG. 2A. It should be understood that the sensor 13 shown is intended to represent one or more sensors. In an embodiment, the one or more sensors 13 include a single sensor of one of the types described below. In another embodiment, the one or more sensors 13 include at least two EEG sensors. In still another embodiment, the one or more sensors 13 include at least two EEG sensors and one or more brain oxygenation sensors, and the like. In each of the foregoing embodiments, additional sensors of different types are also optionally included. Other combinations of numbers and types of sensors are also suitable for use with the physiological monitoring system 10.

In some embodiments of the system shown in FIG. 2A, all of the hardware used to receive and process signals from the sensors are housed within the same housing. In other embodiments, some of the hardware used to receive and process signals is housed within a separate housing. In addition, the physiological monitor 17 of certain embodiments includes hardware, software, or both hardware and software, whether in one housing or multiple housings, used to receive and process the signals transmitted by the sensors 13.

As shown in FIG. 2B, the EEG sensor 13 can include a cable 25. The cable 25 can include three conductors within an electrical shielding. One conductor 26 can provide power to a physiological monitor 17, one conductor 28 can provide a ground signal to the physiological monitor 17, and one conductor 28 can transmit signals from the sensor 13 to the physiological monitor 17. For multiple sensors, one or more additional cables 15 can be provided.

In some embodiments, the ground signal is an earth ground, but in other embodiments, the ground signal is a patient ground, sometimes referred to as a patient reference, a patient reference signal, a return, or a patient return. In some embodiments, the cable 25 carries two conductors within an electrical shielding layer, and the shielding layer acts as the ground conductor. Electrical interfaces 23 in the cable 25 can enable the cable to electrically connect to electrical interfaces 21 in a connector 20 of the physiological monitor 17. In another embodiment, the sensor 13 and the physiological monitor 17 communicate wirelessly.

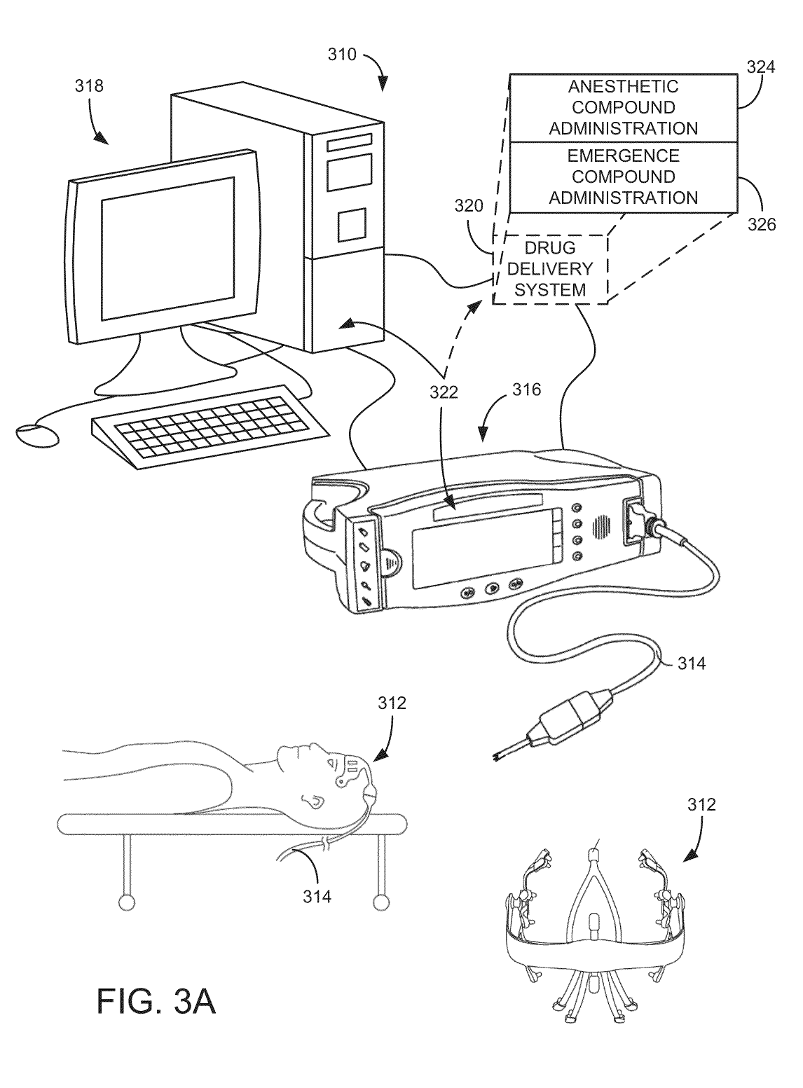

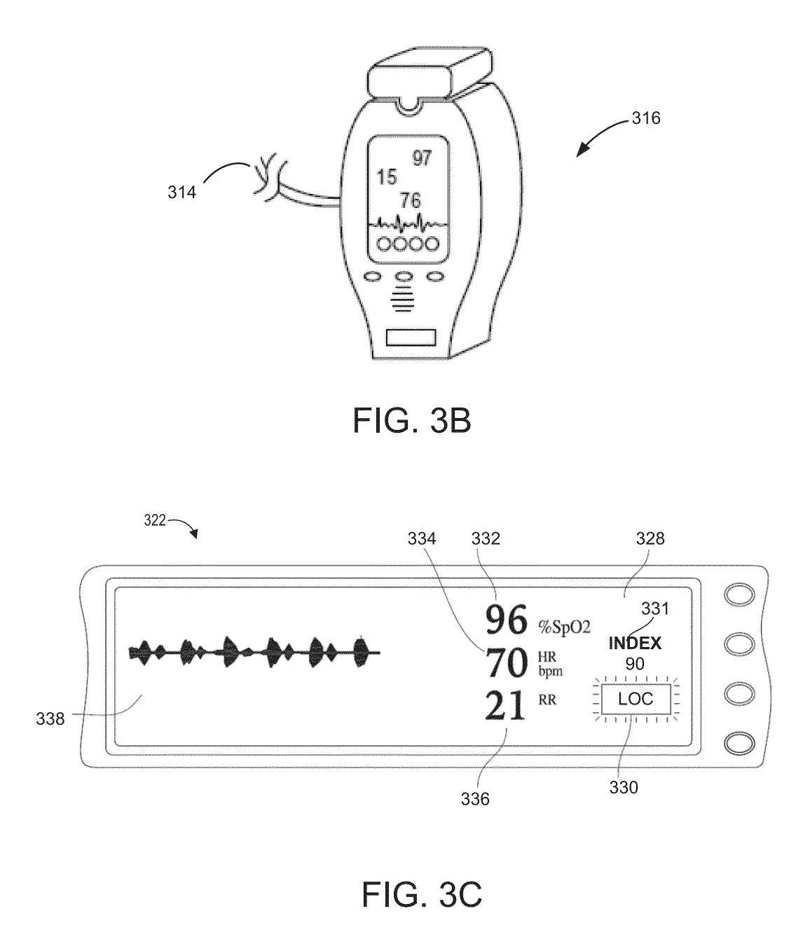

Specifically referring to FIG. 3A, an example system 310 in accordance with the present disclosure is illustrated, for use in monitoring and/or controlling a state of a patient during and after administration of an anesthetic compound or compounds, such as dexmedetomidine. The system 310 includes a patient monitoring device 312, such as a physiological monitoring device, illustrated in FIG. 3 as an electroencephalography (EEG) electrode array. However, it is contemplated that the patient monitoring device 312 may also include mechanisms for monitoring galvanic skin response (GSR), for example, to measure arousal to external stimuli or other monitoring system such as cardiovascular monitors, including electrocardiographic and blood pressure monitors, and also ocular Microtremor monitors. One specific realization of this design utilizes a frontal Laplacian EEG electrode layout with additional electrodes to measure GSR and/or ocular microtremor. Another realization of this design incorporates a frontal array of electrodes that could be combined in post-processing to obtain any combination of electrodes found to optimally detect the EEG signatures described earlier, also with separate GSR electrodes. Another realization of this design utilizes a high-density layout sampling the entire scalp surface using between 64 to 256 sensors for the purpose of source localization, also with separate GSR electrodes.

The patient monitoring device 312 is connected via a cable 314 to communicate with a monitoring system 316, which may be a portable system or device (as shown in FIG. 3B), and provides input of physiological data acquired from a patient to the monitoring system 316. Also, the cable 314 and similar connections can be replaced by wireless connections between components. As illustrated, the monitoring system 316 may be further connected to a dedicated analysis system 318. Also, the monitoring system 316 and analysis system 318 may be integrated.

The monitoring system 316 may be configured to receive raw signals acquired by the EEG electrode array and assemble, and even display, the raw signals as EEG waveforms. Accordingly, the analysis system 318 may receive the EEG waveforms from the monitoring system 316 and, as will be described, analyze the EEG waveforms and signatures therein based on a selected anesthesia compound, determine a state of the patient based on the analyzed EEG waveforms and signatures, and generate a report, for example, as a printed report or, preferably, a real-time display of signature information and determined state or index. However, it is also contemplated that the functions of monitoring system 316 and analysis system 318 may be combined into a common system. In one aspect, the monitoring system 316 and analysis system 318 may be configured to determine, based on measures, such as activity rate, power, amplitude, and so forth, associated with transient and low frequency oscillations, a current and future brain state under administration of anesthetic compounds, or target endpoint, such as during general anesthesia or sedation.

In some configurations, the system 310 may also include a drug delivery system 320. The drug delivery system 320 may be coupled to the analysis system 318 and monitoring system 316, such that the system 310 forms a closed-loop monitoring and control system. Such a closed-loop monitoring and control system in accordance with the present disclosure is capable of a wide range of operation, and may include a user interface 322, or user input, to allow a user to configure the closed-loop monitoring and control system, receive feedback from the closed-loop monitoring and control system, and, if needed reconfigure and/or override the closed-loop monitoring and control system.

The system 310 can include or be coupled to a drug delivery system 320 with two specific sub-systems. As such, the drug delivery system 320 may include an anesthetic compound administration system 324 that is designed to deliver doses of one or more anesthetic compounds to a subject and may also include a emergence compound administration system 326 that is designed to deliver doses of one or more compounds that will reverse general anesthesia or the enhance the natural emergence of a subject from anesthesia.

Referring specifically to FIG. 3C, a non-limiting example user interface 322 is illustrated, including a multiparameter physiological monitor display 328. The display 328 can output a loss of consciousness (“LOC”) indicator 330 or, as will be described, an index 331. The loss of consciousness indicator 330 can be generated using any of the techniques described herein. The display 328 may also include parameter data for SpO2 332, and pulse rate 334 in beats per minute (“BPM”), and rate of respiration (“RR”) indicator 336. In the depicted embodiment shown in FIG. 3B, the LOC indicator 330 includes text that indicates that the patient has lost consciousness. In some embodiments, an index 331 may be include that indicates a state of consciousness, or degree of sedation, of the patient. For example, the index 331 may range from 0 to 100. A light sedation may be indicated by an index of 75, while a deep sedation may indicated by an index of 50, although other values are possible. In some embodiments, the index 331 is a function of confidence. Other factors (e.g. spindle rate, determined power in particular frequency bands, signature correlation) may also be used to calculate an index or brain state. The text displayed in the LOC indicator 330 may depend on a confidence calculation from one of the consciousness state detection processes described herein. Each one of the consciousness state detection processes described above may have different confidence rating depending on how accurately the particular process or combination of processes can predict a state of consciousness condition. The confidence rating may be stored in the patient monitor. In some embodiments, more than one of processes (described above) can be used to determine the LOC indicator 330. Furthermore, the display 328 can output any segment of raw or processed waveform signals 330, including EEG signals or spectrograms intermittently or in real time.

Referring back to FIG. 3A, in some configurations, the drug delivery system 320 is not only able to control the administration of anesthetic compounds for the purpose of placing the patient in a state of reduced consciousness influenced by the anesthetic compounds, such as general anesthesia or sedation, but can also implement and reflect systems and methods for bringing a patient to and from a state of greater or lesser consciousness.

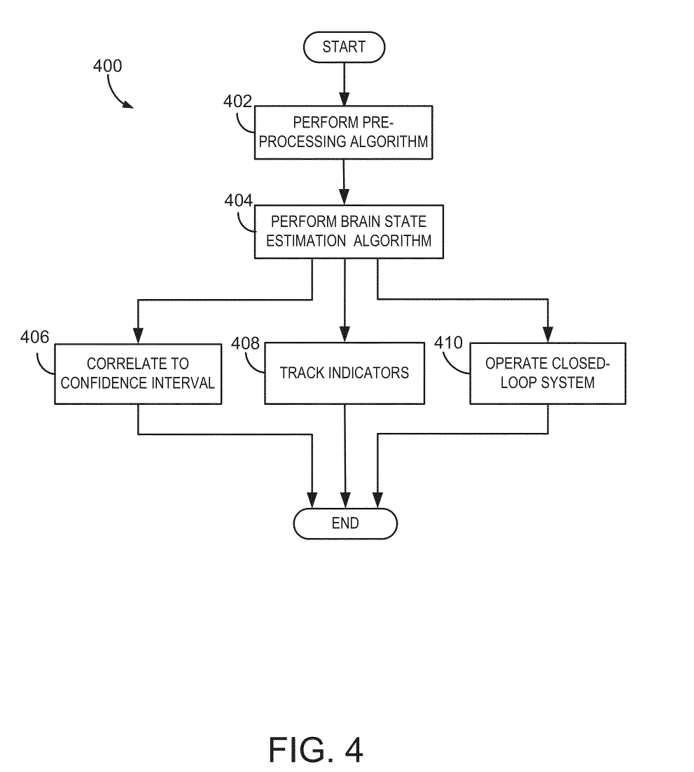

Turning now to FIG. 4, a process 400 in accordance with the present disclosure begins at process block 402 by performing a pre-processing algorithm that analyzes waveforms from an EEG monitoring system. At this step the raw EEG data may be modified, transformed, enhanced, filtered, or manipulated to take any desired or required form, or possess any desired or required features or characteristics. For example, the raw EEG data may be assembled into time-series data or waveforms. U.S. Provisional Application Ser. No. 61/815,606, filed Apr. 24, 2013, and entitled “A METHOD FOR ESTIMATING HIGH TIME-FREQUENCY RESOLUTION EEG SPECTROGRAMS TO MONITOR GENERAL ANESTHESIA AND SEDATION,” is incorporated herein by reference in its entirety.

Moreover, at process block 402, indicators related to the EEG data or waveforms may be identified, or determined, including indicators related to target wave or non-transient oscillations (for example, slow/delta frequency oscillations in the range between 0.1 and 6 Hz) and transient oscillations (for example, oscillations or “spindles” in the range between 12 and 16 Hz) present in the EEG waveforms. For example, the indicators may reflect specific oscillation signatures such as occurrence rates, as in the case of transient oscillations, as well as other target wave signatures or characteristics, such as power spectra characteristics, amplitude characteristics and so forth, for slow/delta frequency oscillations.

The pre-processed data is then, at process block 404, provided as an input into a brain state estimation algorithm. In one aspect, the brain state estimation algorithm may perform a determination of current and/or future depth of sedation related to physiological data measures, under administration of any combination of anesthetic compounds, such as during sedation using dexmedetomidine.

The brain state estimation algorithm output, at process block 406, may be correlated with “confidence intervals.” The confidence intervals are predicated on formal statistical comparisons between the brain state estimated at any two time points. Also, at process block 408, the output of the brain state estimation algorithm can be used to identify and track brain state indicators, such as depth of sedation by way of transient oscillation, or spindle, and low frequency, such as slow wave or delta wave, oscillation characteristics or signatures, including power spectra, amplitude characteristics, occurrence rates, and so forth, during medical procedures or disease states. Exemplary medically-significant states include general anesthesia, sedation, light sedation, and deep sedation to name but a few. The output of the brain state estimation algorithm may also be used, at process block 410 as part of a closed-loop anesthesia control process.

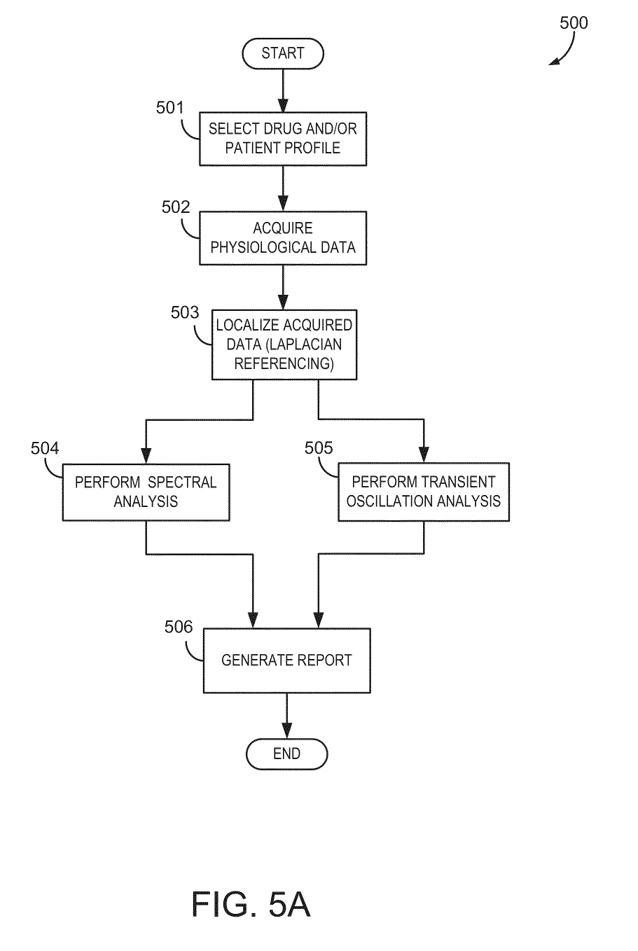

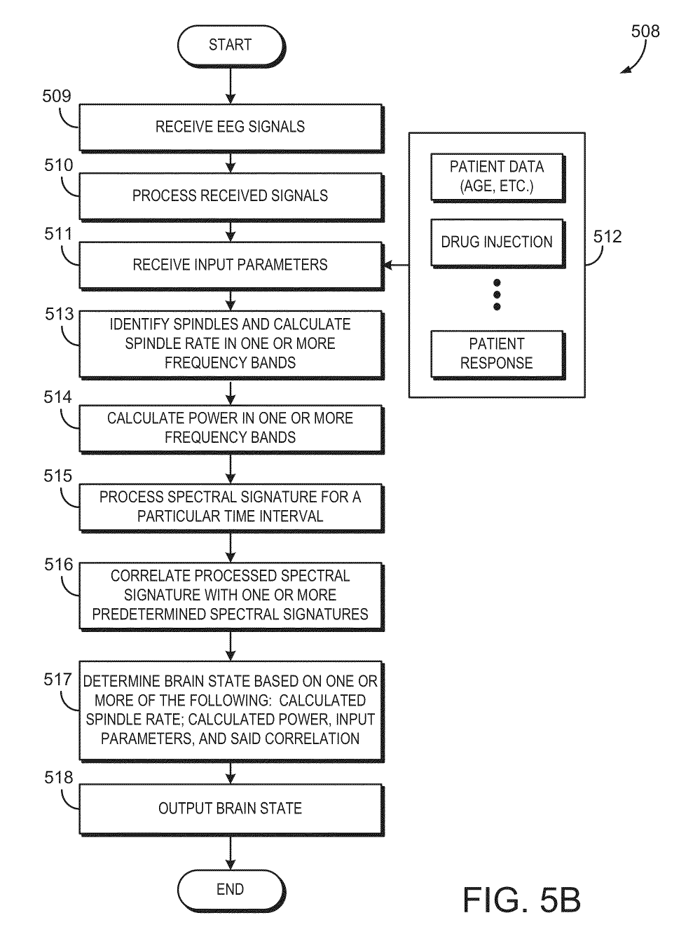

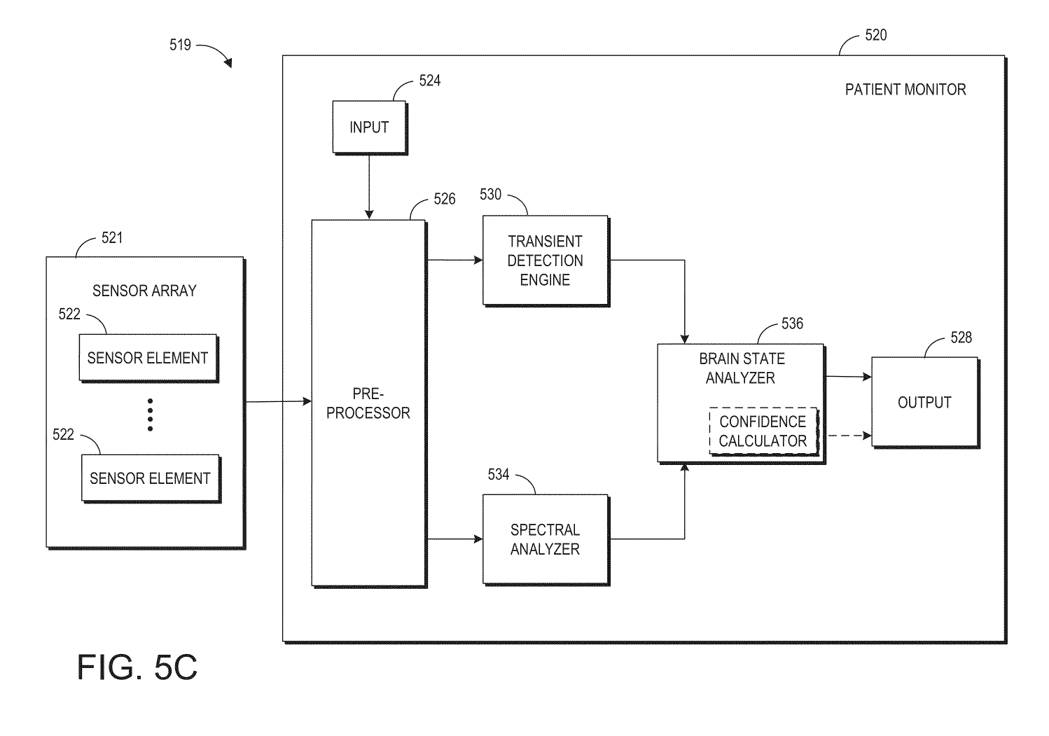

In another embodiment, the present disclosure provides a method for monitoring and control in accordance with the present invention. Referring now to FIG. 5A, the process 500 begins at process block 501 with the selection or indication of a desired drug, such as anesthesia compound or compounds, and/or a particular patient profile, such as a patient's age height, weight, gender, or the like. Furthermore, drug administration information, such as timing, dose, rate, and the like, in conjunction with the above-described EEG data may be acquired and used to estimate and predict future patient states in accordance with the present invention. As will be described, the present invention recognizes that the physiological responses to anesthesia vary based on the specific compound or compounds administered, as well as the patient profile. For example, elderly patients have a tendency to show lower amplitude alpha power under anesthesia, with some showing no visible alpha power in the unconscious state. The present disclosure accounts for this variation between an elderly patient and a younger patient. Furthermore, the present disclosure recognizes that analyzing physiological data for signatures particular to a specific anesthetic compound or compounds administered and/or the profile of the patient substantially increases the ability to identify particular indicators of the patient's brain being in a particular state and the accuracy of state indicators and predictions based on those indicators.

For example, the following drugs are examples of drugs or anesthetic compounds that may be used with the present invention: Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Nitrous oxide, Xenon, Remifenanil, Fentanyl, Sufentanil, Alfentanil, Hydromorphone, and the like. However, the present invention recognizes that each of these drugs, induces very different characteristics or signatures, for example, within EEG data or waveforms. Spindle activity can be observed with these drugs as well however, and could be used to identify sedative states with these drugs also.

With the proper drug or drugs and/or patient profile selected, acquisition of physiological data begins at process block 502, for example, using a system such as described with respect to FIG. 3, where the acquired data is EEG data. The present disclosure provides systems and methods for analyzing acquired physiological information from a patient, analyzing the information and the key indicators included therein, and extrapolating information regarding a current and/or predicted future state, or target endpoint, of the patient. To do so, rather than evaluate physiological data in the abstract, the physiological data is processed. Processing can be done in the electrode or sensor space or extrapolated to the locations in the brain. As will be described, the present invention enables the tracking of the spatiotemporal dynamics of the brain by combining additional analysis tools, including, for example, spectrogram, transient oscillation analysis and so forth. As will be apparent, reference to “spectrogram” may refer to a visual representation of frequency domain information.

At process block 503, Laplacian referencing can be performed to estimate radial current densities perpendicular to the scalp at each electrode site of, for example, the monitoring device of FIG. 3. This may be achieved by taking a difference between voltages recorded at an electrode site and an average of the voltage recorded at the electrode sites in a local neighborhood. Other combinations of information across the plurality of electrodes may also be used to enhance estimation of relevant brain states. In this manner, generated signals may be directly related to electrodes placed on a subject at particular sites, such as frontal, temporal, parietal locations, and so forth, or may be the result of combinations of signals obtained from multiple sites.

Next, at process blocks 504 and 505, different analyses may be performed either independently, or in any combination, to yield any of spectral, temporal, transient, or amplitude related to different spatiotemporal activities at different states of a patient receiving at least one anesthetic drug. In some aspects, information related to a present or future degree, or depth, of sedation, as resulting from, for example, administration of dexemedetomine, may be identified in relation to determined signatures from low frequency oscillations and transient oscillations, along with indications provided by a user, such as administered dose or dose rate. Moreover, a probability of response to a stimulus, such as an auditory, verbal stimulus, or somatosensory stimulus may also be determined using the degree of sedation.

Specifically, at process block 504, spectrograms may be generated and processed, to yield information related to the time variation of relative power of EEG signal data for a range of different frequencies, as shown in the example of FIG. 1. Although spectrogram generation and processing is performed at process block 504, a visual representation of the spectrograms need not be displayed. In some aspects, spectrograms could be generated using multitaper and sliding window methods to achieve precise and specific time-frequency resolution and efficiency, which are properties necessary to estimate the relevant brain states. Again, U.S. Provisional Application Ser. No. 61/815,606 is incorporated herein by reference in its entirety. In other aspects, state-space models of dynamic spectra may be applied to determine the spectrograms, whereby the data drives the optimal amount of smoothing. With respect to determining a degree of sedation as a result of administration of dexmedetomidine, power characteristics may be desirable in a slow/delta wave frequency range (for example, 0.1 to 6 Hz) and a transient oscillation, or sigma, frequency range (for example, 12 to 16 Hz), although other frequency bands may be used.

At process block 505, a transient oscillation analysis may be performed that includes identifying transient oscillation events in the acquired physiological data. In some preferred aspects, transient oscillations, or spindles, may be determined and characterized at process block 505 using a transient oscillation detection technique, similar to a NREM sleep spindle detection technique, although other methods may be possible. Specifically, the transient oscillation technique includes projecting any segment of acquired time-series EEG signals onto a pre-determined basis, defined by a series of eigenfunctions (which may be generated using a pool of waveform data), to generate a set of expansion coefficients for use in evaluating probabilities related to the occurrence of a transient oscillation, or spindle, event. Using a Bayesian approach, the detection technique may then compute a posterior probability indicative of the signals belonging to a transient oscillation event. As a result, at process block 505, a transient oscillation rate, or spindle rate, can be determined along with other transient oscillation characteristics.

The above-described selection of an appropriate analysis context based on a selected drug or drugs (process block 501), the acquisition of data (process block 502), and the analysis of the acquired data (process blocks 504 and 505) set the stage for the new and substantially improved real-time analysis and reporting on the state of a patient's brain as an anesthetic, such as dexmedetomidine, is being administered. That is, although, as explained above, particular indications or signatures related to the states of effectiveness of an administered anesthetic compound or anesthetic compounds can be determined from each of the above-described analyses (particularly, when adjusted for a particular selected drug or drugs), the present disclosure provides a mechanism for considering each of these separate pieces of data and more to accurately indicate and/or report on a state of the patient under anesthesia and/or the indicators or signatures that indicate the state of the patient under anesthesia.

Referring to FIG. 5B, a further example of a process 508 in accordance with the present disclosure begins at process block 509 by receiving EEG signals. At process block 510 the received signals are processed. For example, as described herein, the raw EEG signals may be assembled into a time-series of signals or waveform. Also, at process block 511, input parameters are received. As illustrated at input block 512, some examples of input parameters may include patient data, such as age, gender, weight, drug use history, and the like. Also, the input parameters may include drug information, such as the type or amount of drug delivered to the patient and/or the planned drug to be delivered. Further parameters may include patent response information and the like.

At process block 513, spindles are identified and a spindle rate in one or more frequency bands may be calculated and at process block 514 the power in one or more frequency bands may be calculated. For example, as described above, frequency bands of spectrograms may be analyzed to determine spindle rates and/or power information. For example, as shown FIG. 1A, observed features include a combination of low frequency oscillations 1 (with frequencies less than 6 Hz) and “spindles” 1, or spindle events, which are transient oscillations, generally in a frequency range of 9 to 16 Hz that occur in bursts lasting 1-2 seconds (FIG. 1B). As will be described with respect to FIG. 5C, this may be performed using a combination of electronics and software.

At process block 515, the above-described data may be analyzed to determined any of a variety of spectral signatures, for example, over a particular time interval. For example, again referring to the spectrogram of FIG. 1A, a signature may be spindles 2 that appear as streaks in the high alpha (9-12 Hz) and low beta (13-25 Hz) bands. At process block 516, any spectral signatures may be correlated with predetermined spectral signatures. For example, the predetermined spectral signals may be selected or correlated with the input parameters. For example, referring again to FIG. 1A, a predetermined signature for dexmedetomidine may indicate that spindles often appear as streaks in the high alpha (9-12 Hz) and low beta (13-25 Hz) bands occurring in a similar frequency range as alpha oscillations generated during propofol-induced anesthesia, but with much less power than alpha oscillations. Thus, it can be determined at process block 516 that the spectral signature of FIG. 1A correlates with a predetermined spectral signature for dexmedetomidine.

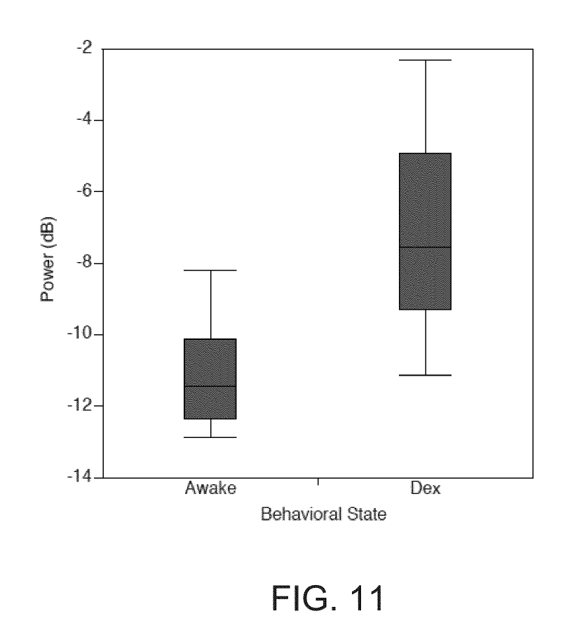

At process block 517, a current or future brain state may be determined using one or more of, for example, calculated spindle rate, calculated power, input parameters, and spectral signature correlation with predetermined spectral signatures. For example, as explained herein in FIGS. 1 and 11, when the rate of dexmedetomidine infusion is increased, spindles disappear and the amplitude of low frequency oscillations increase. Thus, at process block 517, if such pattern is determined, and the input parameters indicate the drug being delivered is dexmedetomidine, a report may be output at process block 518 indicating a current or impending deeper state of sedation.

Referring to FIG. 5C, an example system 519 for carrying out steps for determining a brain state of a patient, as described above, is illustrated. The system 519 includes patient monitor 520 and a sensor array 521 configured with any number of sensors 522 designed to acquire physiological data, such as EEG data. The sensor array 521 is in communication with the patient monitor 520 via a wired or wireless connection.

The patient monitor 520 is configured to receive and process data provided by the sensor array 522, and includes an input 524, a pre-processor 526 and an output 528. In particular, the pre-processor 526 is configured to carry out any number of pre-processing steps, such as assembling the received physiological data into time-series signals and performing a noise rejection step to filter any interfering signals associated with the acquired physiological data. The pre-processor is also configured to receive an indication via the input 524, such as information related to administration of an anesthesia compound or compounds, and/or an indication related to a particular patient profile, such as a patient's age, height, weight, gender, or the like, as well as drug administration information, such as timing, dose, rate, and the like. The patient monitor 520 further includes a number of processing modules in communication with the pre-processor 526, including a transient detection engine 530, and a spectral analyzer 534. The processing modules are configured to receive pre-processed data from the pre-processor 526 and carry out steps necessary for determining a brain state, such as a degree of sedation, of a patient, as described, which may be performed in parallel, in succession or in combination. Furthermore, the patient monitor 520 includes a brain state analyzer 536 which is configured to received processed information, such as information related to transient and slow/delta wave oscillations, from the processing modules and provide a determination related to a present or future state, or degree of sedation, of a patient under anesthesia and confidence with respect to the determined state(s). Information related to the determined state(s) may then be relayed to the output 528, along with any other desired information, in any shape or form. For example, the output 528 may include a display configured to provide a loss of consciousness indicator, a degree of sedation indicator, a confidence indicator, a probability of response indicator, and so forth, either intermittently or in near real-time, for example, with a latency ranging from hundreds of milliseconds to tens of seconds.

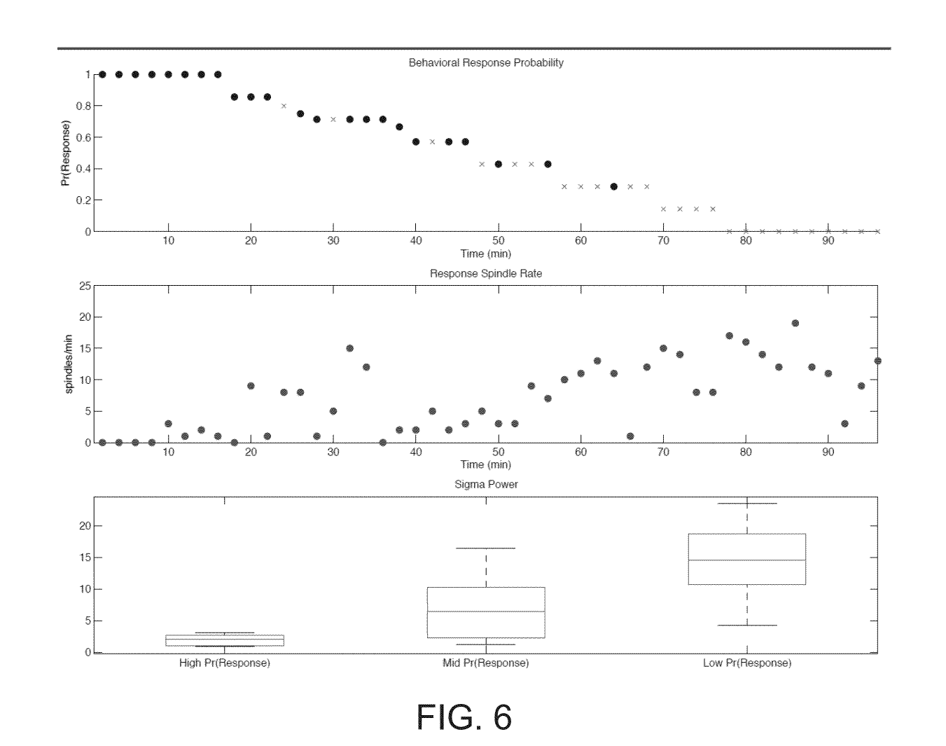

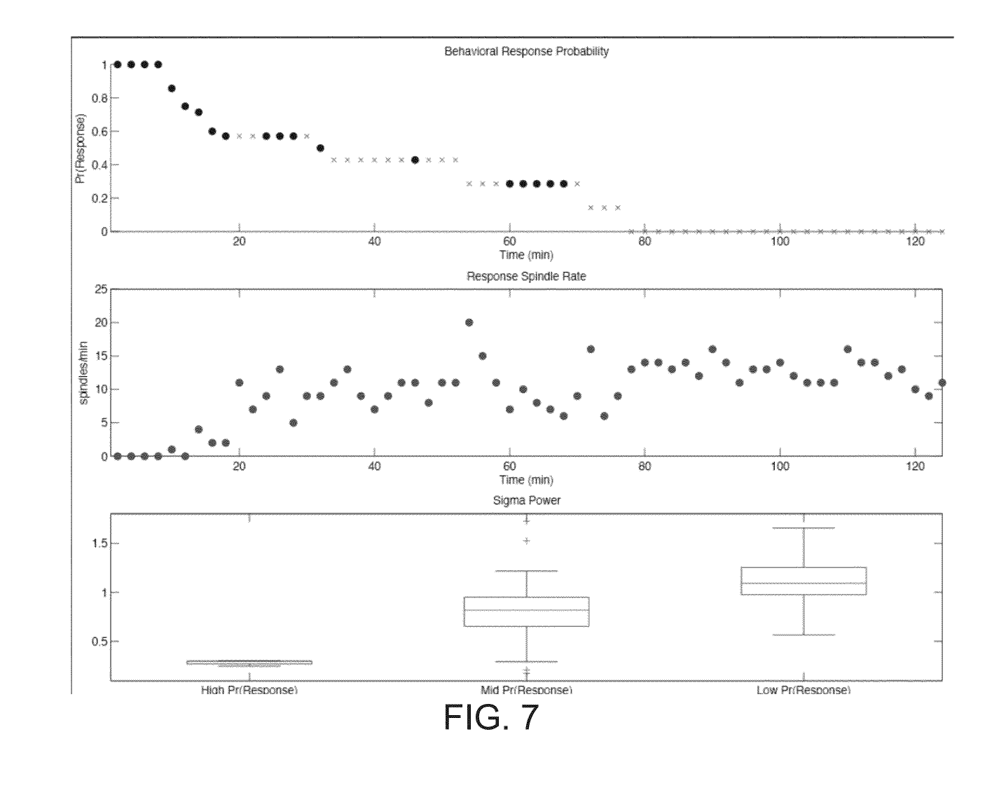

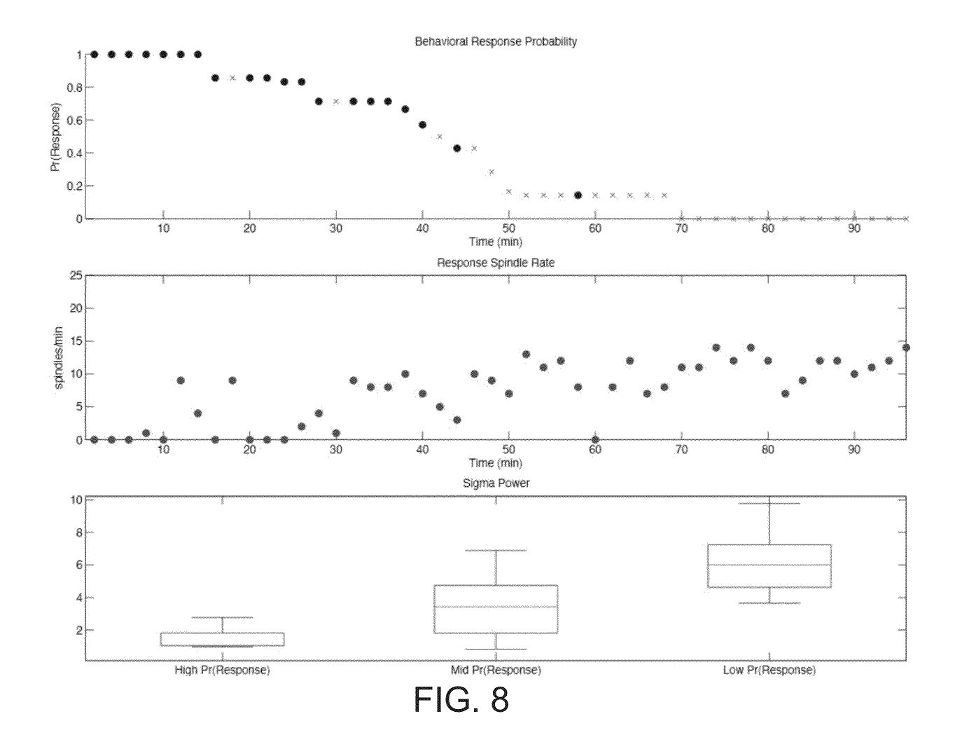

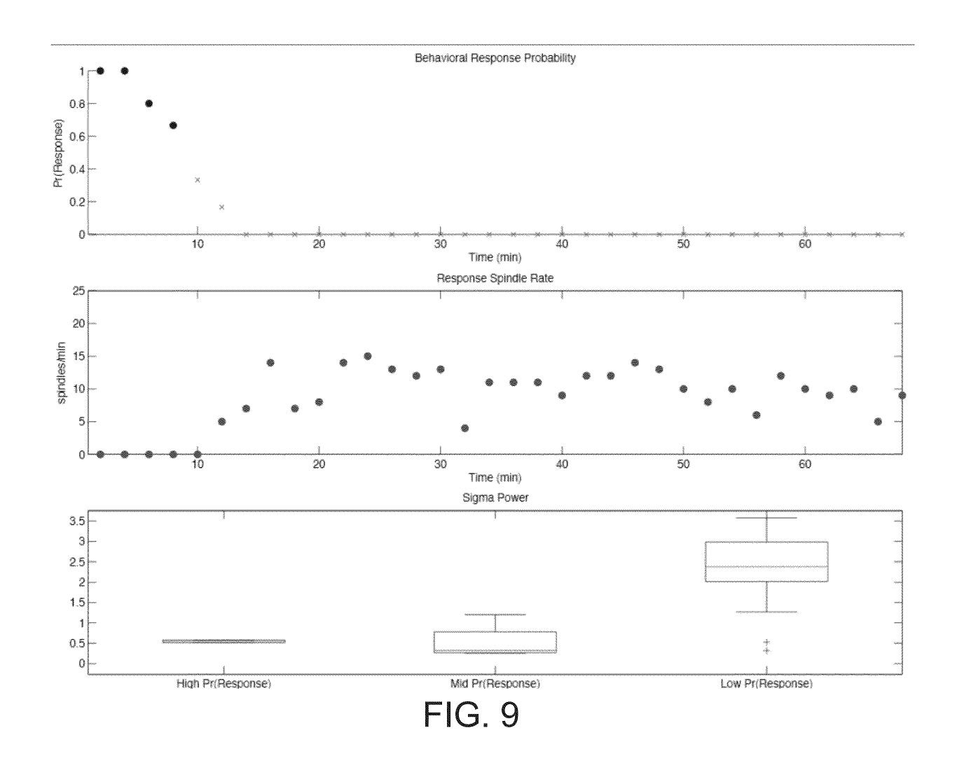

Specifically referring to FIGS. 6-9, graphical examples are shown indicating relationships between probability of response to auditory stimuli (top panel), spindle rate (middle panel), and spindle (sigma, 12-16 Hz) power (lower panel) for EEG data acquired from subjects undergoing dexmedetomidine sedation. Each subject was administered a 1 mcg/kg loading bolus of dexmedetomidine over 10 minutes, starting approximately at the 10 minute mark, followed by a 0.7 mcg/kg/hr maintenance dose of dexmedetomidine. The drug effects were quantified in the top panels in terms of probability of response. The individual responses and non-responses to auditory stimuli were distinguished in the figures by the “o” and “x” symbols, respectively. As is appreciated from the figures, as the drug takes effect, a subject becomes increasingly sedated, which is reflected in the decrease in the probability of response. At the same time, the spindle rate and spindle power increase. Spindle power shown in FIGS. 6-9 was calculated for three conditions: High probability of response (>=90%), Medium probability of response (<90% and >=10%), and Low probability of response (<10%). As shown in FIG. 11, a spectral analysis of the slow/delta (0.5-5 Hz) frequency band identifies a statistically significant difference between dexmedetomidine-induced loss of consciousness and the baseline awake state (P>0.0039, Wilcoxon signed-rank test). To estimate the power in each band of interest for each subject, baseline (n=9) and dexmedetomidine-induced unconsciousness (n=9) spectrograms were averaged across the slow/delta frequency band over a 2 minute EEG epoch, obtaining two data points per subject for use in group-level paired data analysis. Data are presented as box plots with the boxes representing the 25th to 75th percentiles, the lines within the boxes showing the median. Thus, slow/delta (0.5-5 Hz) power is larger after loss of consciousness

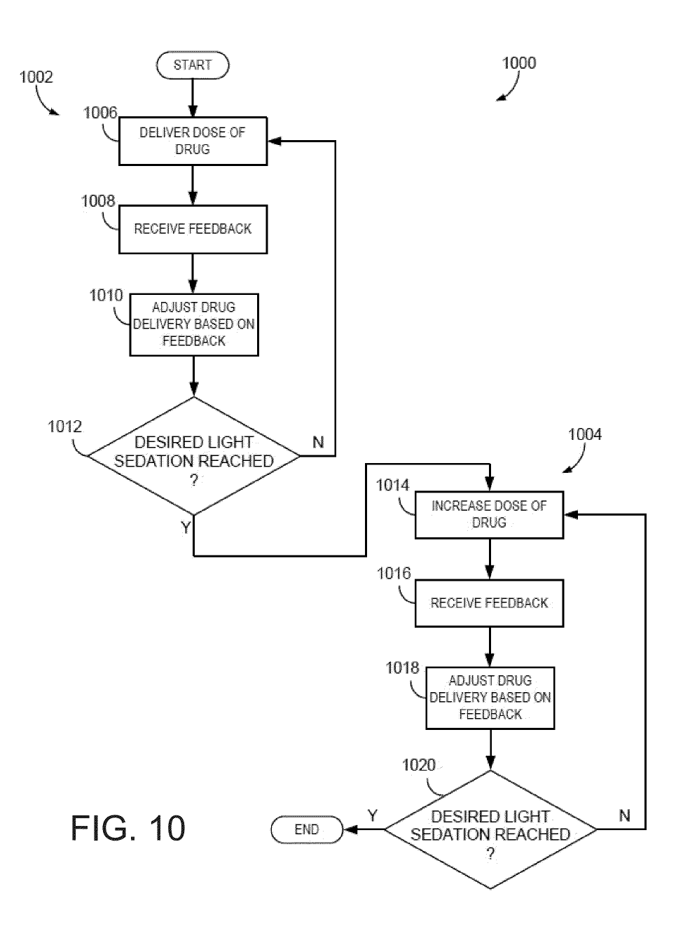

As a non-limiting example, referring to FIG. 10, example steps 1000 for a clinical case are provided. As will be described, in this non-limiting example, a light sedation is desired during a first portion of the process 1002 and a deeper level is desired during a second portion of the process 1004. During the first portion of the process 1002 where light sedation is desired, an initial amount of drug is delivered to the patient at process block 1006. At process block 1008, feedback is received to determine the level of sedation that has been reached. The feedback may be both qualitative or subjective and quantitative or objective feedback. At a basic level, with light sedation, qualitative or subjective feedback may be gathered using verbal commands or somatosensory stimuli to arouse or to solicit feedback from the patient. In addition, quantitative or objective feedback may be gathered regarding light sedation by evaluating a spindle rate 2, such as illustrated in FIG. 1A. In particular, such quantitative feedback may be provided using monitoring systems, as described in FIG. 5C, whereby transient oscillation and spectral information from processed physiological data may be used to determine a brain state of a subject, in accordance with the present disclosure.

Using the feedback from process block 1008, the drug delivery may be adjusted at process block 1010. For example, the infusion of dexmedetomidine could be adjusted to a level where both spindles 2 and slow/delta waves 1 of FIG. 1A are present with a spindle rate between 10 and 15 spindles per minute, as also shown in FIGS. 6, 7, 8, and 9. At decision block 1012, a check is made to determine whether the desired level of light sedation has been reached. If not, the process repeats. If so, in this example, the underlying medical process may continue to the second portion of the process 1004 where a deeper level of sedation is desired.

At process block 1014, the drug dose is increased toward a deeper level of sedation. At process block 1016, feedback is received to determine the level of sedation that has been reached. Again, the feedback may be both qualitative or subjective and quantitative or objective feedback. At a basic level, with deeper sedation, qualitative or subjective feedback may not be as readily gathered using verbal commands or somatosensory stimuli to arouse or to solicit feedback from the patient. In addition, quantitative or objective feedback may be gathered regarding deeper sedation by evaluating a spindle rate 2 and slow/delta waves 1 as shown FIG. 1A.

Using the feedback from process block 1016, the drug delivery may be adjusted at process block 1018. For example, the infusion of dexmedetomidine could be adjusted to a level where spindles 2, such as illustrated in FIG. 1A, decrease and stop appearing and only slow/delta waves 1 of FIG. 1A are present. In particular, in this example, deep sedation may be determined when only strong slow waves were observed, as in FIG. 1C and FIG. 11. At decision block 1020, a check is made to determine whether the desired level of deep sedation has been reached. If not, the process repeats. If so, in this example, the process ends.

Referring again to FIG. 5A, at process block 506, any and all of the above-described analysis and/or results can be combined and reported, in any desired or required shape or form, including providing a report in real time, and, in addition, can be coupled with a precise statistical characterizations of behavioral dynamics, for use by a clinician or use in combination with a closed-loop system as described above. In particular, behavioral dynamics, such as the points of loss-of-consciousness, degree of sedation and recovery-of-consciousness can be precisely, and statistically calculated and indicated in accordance with the present disclosure. In some aspects, the report may include a probability of response to at least one of an auditory stimulus, a verbal stimulus and a somatosensory stimulus.

Embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. In addition, the foregoing embodiments have been described at a level of detail to allow one of ordinary skill in the art to make and use the devices, systems, etc. described herein. A wide variety of variation is possible. Components, elements, and/or steps can be altered, added, removed, or rearranged. While certain embodiments have been explicitly described, other embodiments will become apparent to those of ordinary skill in the art based on this disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

1. A system for monitoring a patient experiencing an administration of at least one drug having anesthetic properties, the system comprising:
an input configured to receive physiological data from at least one sensor coupled to the patient;
at least one processor configured to:
receive the physiological data from the input;
assemble the physiological data into sets of time-series data;
determine, from the sets of time-series data, a first set of signals in a first frequency range and a second set of signals in a second frequency range, the first set of signals describing a transient oscillation signature and the second set of signals describing a target wave signature;
identify, using the transient oscillation and target wave signatures, a degree of sedation consistent with the administration of at least one drug having anesthetic properties; and
generate a report indicative of the degree of sedation induced by the at least one drug having anesthetic properties.

2. The system of claim 1, wherein the first frequency range comprises a frequency range between 12 Hz and 16 Hz.

3. The system of claim 1, wherein the second frequency range comprises a frequency range between 0.1 to 6 Hz.

4. The system of claim 1, wherein the transient oscillation signature is defined by at least one of an activity rate, a sigma power, and an amplitude.

5. The system of claim 1, wherein the target wave signature is defined by at least one of a slow wave power, a slow wave amplitude, a delta wave power, and a delta wave amplitude.

6. The system of claim 1, wherein the at least one processor is further configured to determine the first set of signals using a transient oscillation detection technique.

7. The system of claim 6, wherein the transient oscillation technique comprises projecting the sets of time-series data onto a pre-determined basis defined by a series of eigenfunctions, and computing posterior probabilities indicative of signals belonging to a transient oscillation event.

8. The system of claim 1, wherein the at least one processor is further configured to determine a probability of response to at least one of an auditory stimulus, a verbal stimulus and a somatosensory stimulus using the degree of sedation.

9. The system of claim 1 wherein the at least one processor is further configured to receive from the input an indication comprising a characteristic of the patient, a drug selecting from the list consisting essentially of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and drug administration information including at least one of drug timing, drug dose, drug administration rate, and target endpoint.

10. The system of claim 9, wherein the at least one processor is further configured to guide administration of the at least one drug having anesthetic properties to a target endpoint, using the degree of sedation and the indication.

11. A method for monitoring a patient experiencing an administration of at least one drug having anesthetic properties, the method comprising:
arranging at least one sensor configured to acquire physiological data from a patient;
reviewing the physiological data from the at least one sensor and an indication received from an input;
assembling the physiological data into sets of time-series data;
determining, from the sets of time-series data, a first set of signals in a first frequency range and a second set of signals in a second frequency range, the first set of signals describing a transient oscillation signature and the second set of signals describing a target wave signature;
identifying, using the transient oscillation and target wave signatures, a degree of sedation consistent with the administration of at least one drug having anesthetic properties; and
generating a report indicative of the degree of sedation induced by the at least one drug having anesthetic properties.

12. The method of claim 11, wherein the first frequency range comprises a frequency range between 12 Hz and 16 Hz.

13. The method of claim 11, wherein the second frequency range comprises a frequency range between 0.1 to 6 Hz.

14. The method of claim 11, wherein the transient oscillation signature is defined by at least one of an activity rate, a sigma power, an amplitude.

15. The method of claim 11, wherein the target wave signature is defined by at least one of a slow wave power, a slow wave amplitude, a delta wave power, and a delta wave amplitude.

16. The method of claim 11, wherein the method further comprises determining the first set of signals using a transient oscillation detection technique.

17. The system of claim 16, wherein the transient oscillation technique comprises projecting the sets of time-series data onto a pre-determined basis defined by a series of eigenfunctions, and computing posterior probabilities indicative of signals belonging to a transient oscillation event.

18. The method of claim 11, wherein the method further comprises determining a probability of response to at least one of an auditory stimulus, a verbal stimulus and a somatosensory stimulus using the degree of sedation.

19. The method of claim 11 wherein the indication comprises a characteristic of the patient, a drug selecting from the list consisting essentially of Propofol, Etomidate, Barbiturates, Thiopental, Pentobarbital, Phenobarbital, Methohexital, Benzodiazepines, Midazolam, Diazepam, Lorazepam, Dexmedetomidine, Ketamine, Sevoflurane, Isoflurane, Desflurane, Remifenanil, Fentanyl, Sufentanil, Alfentanil, and drug administration information including at least one of drug timing, drug dose, drug administration rate, and target endpoint.

20. The method of claim 19, wherein the method further comprises guiding administration of the at least one drug having anesthetic properties to the target endpoint using the degree of sedation and the indication.
